(19) United States
(12) Patent Application Publication
(10) Pub. No.: US 2004/0234571 A1
(43) Pub. Date: Nov. 25, 2004

(54) Direct injection of nano fibers and nano fiber composites for biomedical applications

(76) Inventor: Bor Z. Jang, (US)

Correspondence Address: Bor Z Jang, 2902, 28 AVE, S.W., FARGO, ND, 58103, US

(21) Appl. No.: 10/442,561

(22) Filed: May 22, 2003

(51) Int. Cl. A61K 45/00; B29C 35/08

(52) U.S. Cl. 424/423; 424/93.7; 264/494

(57) ABSTRACT

A process for injecting nanometer-scaled fibers directly into an intended body site of a patient. The process includes the steps of (a) preparing a precursor fluid to the fibers and (b) injecting the precursor fluid into the intended body site under the influence of an electrical field established between two electrodes to produce the nanometer-scaled fibers for forming a reinforcement preform. A polymer is then optionally injected into the intended body site to form a nano fiber-polymer composite structure. The composite structure may contain interconnected macro pores wherein cells can grow and proliferate. This composite scaffold is useful for tissue engineering. The injected nano fibers and composite structure may also be used as a means of controlled drug release or bone reinforcement.

### FIELD OF INVENTION

The present invention relates to a process for directly injecting nanometer-scaled fibers (nano fibers) and nano fiber composites into an intended site in a living body for the purposes of tissue engineering, controlled release of drug, or bone reinforcement. In particular, the invention provides a process for direct injection of nano fibers from a solution or melt of a biodegradable, biocompatible, and/or bioresorbable polymer into an intended living body site. This nano fiber formation step is optionally followed by direct injection of a matrix polymer into the same site to form a nano fiber reinforced polymer composite.

### BACKGROUND

A primary goal of tissue engineering research is the development of effective techniques to repair, replace, or regenerate damaged or diseased tissues by manipulating cells, creating artificial implants, or synthesizing laboratory-grown substitutes. This subject was recently reviewed by D. W. Hutmacher, (“Scaffolds in tissue engineering bone and cartilage,” Biomaterials, 21 (2000) 2529-43). Several approaches to regenerative tissue engineering have been proposed. The “tissue induction” approach involves implanting polymer or mineral scaffolds without cells in a patient. In this process, tissue generation occurs through ingrowth of surrounding tissue into the scaffold. The “cell transplantation” approach involves seeding scaffolds with cells, cytokines, and other growth-related molecules and then culturing and implanting these constructs to induce the growth of new tissue. Cultured cells are infused in a biodegradable or non-biodegradable scaffold, which may be either implanted directly in the patient or be placed in a bio-reactor (in-vitro) to allow the cells to proliferate before the tissue is implanted in the patient. Alternatively, the cell-seeded scaffold may be directly implanted. In this case the patient's body acts as an in-vivo bio-reactor. Once implanted, in-vivo cellular proliferation occurs and, in the case of bio-absorbable scaffolds, concomitant bio-absorption of the scaffold proceeds.

The scaffold, whether or not bio-absorbable, must be bio-compatible, such that it does not induce an adverse immune response from the patient or result in toxicity to the patient. The scaffold must be highly porous with an interconnected pore network for cell growth and flow transport of nutrients and metabolic waste. It must also have suitable surface chemistry for cell attachment, proliferation, and differentiation. Further, the scaffold must have mechanical properties to match those of the tissues at the site of implantation (D. W. Hutmacher, et al. “Mechanical prop. and cell cultural response of polycarbonate scaffolds designed and fabricated via fused deposition modeling,” J. Biomed. Material Res. 55 (2001) 203-216). Numerous techniques are currently available for manufacturing scaffolds for tissue generation. The techniques used are often dictated by the type of tissue ultimately being generated.

One purpose of using a scaffold is to support cells. These cells, after being seeded into the scaffold, cling to the interstices of the scaffold and replicate, produce their own extra-cellular matrices, and organize into the target tissue. In many potential applications (e.g., cartilage, bone and tendon regeneration), mechanical integrity (stiffness and strength) of a scaffold is a critical factor that affects the success or failure of the implanted scaffold. Specifically, in vivo, the scaffold structure should protect the inside of the pore network proliferating cells and their extracellular matrix from being mechanically overloaded for a sufficiently long period of time. However, most of the state-of-the-art techniques and the associated materials used do not provide scaffolds with adequate mechanical integrity. Furthermore, in all of these techniques, a surgical operation is required in order for the pre-fabricated scaffold to be implanted in the body of a patient.

For instance, Vacanti, et al. (U.S. Pat. No. 5,736,372, Apr. 7, 1998) proposed to create new joint surfaces using a synthetic polymeric mesh seeded with chondrocytes, which forms new cartilage as the polymer degrades. Although this is promising, the seeded mesh must still be implanted surgically. Several other research groups have considered direct injection of polymers as a highly promising approach to tissue engineering (e.g., P. Soon-Shiong, et al. “Gel compositions prepared from cross-linkable polysaccharides, polycations and/or lipids and uses therefor,” U.S. Pat. No. 5,700,848 (Dec. 23, 1997); L. Griffith-Cima, et al. “Tissue formation by injecting a cell-polymeric solution that gels in vivo,” U.S. Pat. No. 5,709,854 (Jan. 20, 1998); V. R. Shastri, et al., “Semi-interpenetrating polymer network,” U.S. Pat. No. 5,837,752 (Nov. 17, 1998); J. A. Hubbell, “Injectable hydrogel compositions,” U.S. Pat. No. 6,129,761 (Oct. 10, 2000); R. S. Langer, et al., “Semi-interpenetrating or inter-penetrating polymer networks for drug delivery and tissue engineering,” U.S. Pat. No. 6,224,893 (May 1, 2001)). Again, however, a directly injected polymer matrix alone does not provide adequate mechanical integrity to the scaffold in many application situations. Reinforcement fibers are needed to make a composite of higher strength and stiffness from a matrix polymer.

In a related subject, electro-spinning has been successfully used to produce nano-scaled fibers from a variety of polymers (e.g., Cappello and McGrath, “Spinning of Protein Polymer Fibers” in Silk Polymers: Materials Science and Biotechnology, July 1993, pp. 311-327; Reneker and Chun, “Nanometer Diameter Fibers of Polymer, Produced by Electrospinning” Nanotechnology, 7 (1996) 216-223; Fang and Reneker, “DNA Fibers by Electrospinning” J. Macromol. Sci.-Phys., B36 (2) (1997) 169-173; Liivak et al., “A Microfabricated Wet-Spinning Apparatus to Spin Fibers of Silk Proteins: Structure-Property Correlations” Macromolecules, 31 (1998) 2947-2951; Shin, et al. “Experimental Chracterization of Electrospinning,” Polymer, 42 (2001) 9955-67; S. Zarkoob, et al., “Synthetically spun silk nano fibers and a process for making the same,” U.S. Pat. No. 6,110,590, Aug. 29, 2000). However, this prior-art process involves the use of a bulky collector to receive the produced nano fibers. The fibers are not directly injected into a small cavity site in a patient. In theory, these fibers may be formed into a reinforcement scaffold or a composite scaffold outside the body of a patient, but a surgery procedure would be required in order to implant this scaffold into the body. Furthermore, the prior-art electro-spinning apparatus makes use of a high voltage in order to produce a high electric field strength due to a large inter-electrode spacing.

Hence, it is desirable to develop a technique that is capable of:

(a) forming a net-shape nano fiber- or nano fiber composite-based scaffold that is of good mechanical integrity, preferably without involving a high voltage; and

(b) forming, by direct injection, such a scaffold in situ or in vivo at the intended site in a patient in a minimally invasive fashion, obviating the need for a surgery procedure to implant the scaffold.

The present invention results from a research and development effort aiming at achieving the above primary objectives.

### SUMMARY OF THE INVENTION

Herein reported is a novel and innovative approach to the in situ formation of nano fiber- or nano fiber composite-based scaffolds in an intended body site primarily for tissue engineering. A preferred embodiment of the invented process based on this approach entails electro-spinning of nano fibers directly into an intended cavity site in a patient using a pair of syringe-type electrodes. The process begins with direct injection and spinning of a precursor polymer fluid through a first syringe into the cavity. This first syringe with a metal tip (needle) or metal-coated glass tip, in combination with a second electrode (preferably being another syringe with a metal or metal-coated glass needle electrode), provides a high electric field strength needed for electro-spinning. The two respective syringe electrode tips are inserted into the cavity with the two tips closely spaced. The electro-spun nano fibers are cumulated inside the cavity to form a net-shape reinforcement preform (a non-woven network of nano fibers containing essentially interconnected macro pores) according to the shape of the intended body cavity site. This nano fiber-based preform can be used as a scaffold for tissue engineering or controlled drug release; the latter application being possible if therapeutic drug is attached to or embedded in the fiber.

In another preferred embodiment, one of the electrodes is inserted into the intended body site, but the other is positioned outside the site or outside the patient's body (but sufficiently close to the intended site).

Preferably, a desired amount of a polymer matrix is subsequently introduced into the preform to form a composite using a syringe, which may be one of the two electrode syringes. This step involves injection of a matrix polymer-forming solution into the interstitial pores of the reinforcement preform to form a composite. The matrix polymer serves as a binder to bond nano fibers together at least at their points of contact, still leaving behind macro pores which are preferably interconnected. The nano fibers impart mechanical strength and stiffness to the resulting composite. Such a porous composite, comprising a nano fiber-based reinforcement phase, a polymer matrix phase, and macro pores, can be used as a scaffold for tissue engineering. The matrix polymer-forming solution may be mixed with desired cells (e.g., chondrocytes) to form a polymer-cell suspension prior to being injected. Alternatively, the cells may be injected, through the same or a different syringe, into the composite scaffold after the matrix polymer is injected.

In another preferred embodiment, the injected nano fibers and matrix polymer are allowed to essentially completely fill the intended cavity site to make a solid composite. This pore-free composite is particularly useful for reinforcing a defected or cavitied bone.

The matrix polymer may be a polymer gel, an inter-penetrating network (IPN), or a semi-interpenetrating network (S-IPN). Both the nano fibers and the matrix polymer are preferably bio-compatible, biodegradable and bio-resorbable. They will preferably be bio-degraded and bio-resorbed when and after tissue formation occurs.

This net-shape electro-spinning and direct injection process provides a minimally invasive means of forming in vivo a scaffold of excellent mechanical integrity. This process makes it possible to avoid the surgical operation that is otherwise required for implanting a pre-fabricated scaffold into a patient's body. No post-fabrication trimming of the scaffold is needed since it is formed in a net-shape fashion with the intended cavity site serving as the shaping mold. The scaffold provides a biomimetic environment that is conducive to the growth, proliferation and differentiation of cells. The invented approach is suitable for tissue engineering of a wide range of cell structures, including bone, cartilage, tendon, ligament, nerve, blood vessel, skin, bladder, heart, liver, kidney, and lung.

### BRIEF DESCRIPTION OF THE DRAWINGS

FIG. 1 Schematic of a direct injection apparatus for electro-spinning of nano fibers and direct injection of a matrix polymer into an intended cavity site of a patient's body.

FIG. 2 An alternative configuration for a direct injection apparatus, wherein an optical fiber transmits light waves into the intended body site and one end of the optical fiber is metal-coated to serve as an electrode.

FIG. 3 An alternative configuration for a direct injection apparatus, wherein one electrode is positioned outside of a patient's body.

### DETAILED DESCRIPTION OF PREFERRED EMBODIMENTS

A novel and innovative process for the in situ formation of a nano fiber preform shape or a nano fiber composite in an intended body site of a patient has been developed. The preform or composite, if containing macro pores (pore size greater than 100 μm), can be used primarily as a scaffold for tissue engineering. The preform or composite may serve as a means of controlled drug release or serve to reinforce a weakened or sponge-like bone in a patient.

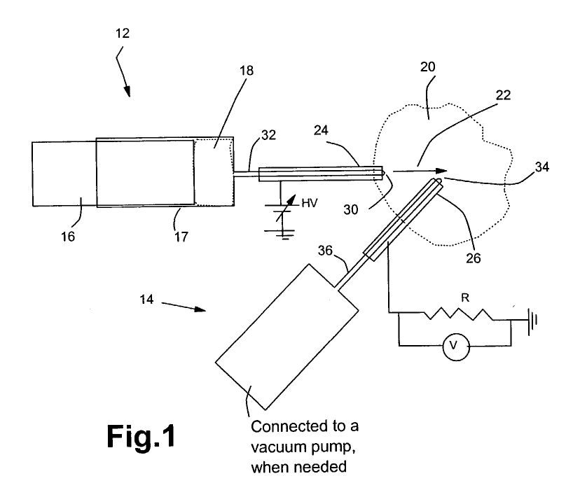

A preferred embodiment of the invented process includes electro-spinning of nano fibers directly into an intended cavity site in a patient's body or an animal body using a pair of syringe-type electrodes. Referring to FIG. 1, the process begins with direct injection of a precursor polymer fluid 18 through a needle 32 and its tip orifice 30 of a first syringe 12 into the cavity 20. In addition to serving as a means of delivering a precursor fluid, this first syringe also serves as an electrode. This first syringe with a metallic needle tip or metal-coated glass tip (metal coating 24 connected to a power supply HV, shown in FIG. 1), in combination with a second electrode (e.g., metal coating 26 on the surface of a glass needle or pipette 36), provide a high electric field strength 22 needed for converting the fluid into nano fibers. The second electrode is preferably another syringe with a metal or metal-coated glass needle, which may be electrically grounded as shown in FIG. 1. The two respective syringe electrode tips are inserted into the cavity with the two tips closely spaced. The electrodes are electrically insulated from the patient. The electro-spun nano fibers are cumulated inside the cavity to form a net-shape reinforcement preform (a non-woven network of nano fibers containing essentially interconnected macro pores) according to the shape of the intended body cavity site.

It is well known that conventional fiber spinning techniques, such as wet spinning, dry-jet wet spinning and dry spinning, produce fibers in the size range of 10 to 100 microns. It is difficult to make nanometer-diameter fibers using conventional spinning processes. In contrast, electro-spinning is known to be effective in producing nano fibers. The diameter of electro-spun fibers is typically one to two orders of magnitude smaller than that of conventionally spun fibers; typically from several to 100 nm.

In one preferred embodiment, the presently invented process comprises electro-spinning as a procedure for converting a precursor fluid to nanometer-scaled fibers (fibers with a diameter smaller than 100 nm). The process of conventional electro-spinning begins with delivering a fine stream of polymeric liquid that, upon proper evaporation of a solvent, yields nano fibers. The fine stream of liquid is produced by pulling a small amount of fiber-forming precursor fluid through space by using relatively strong electrical forces. The apparatus needed to electro-spin the nano fibers typically includes: (a) a liquid dispensing device such as a syringe that delivers a stream or droplets of a fiber-forming fluid to an electric field, (b) a pair of electrodes (typically plate-type, not needle shape) for producing a strong electric field with the voltage required being typically of 24-30 kV in the state-of-the-art electro-spinning process, and (c) a collection device for capturing the produced nano fibers.

The electric field should be strong enough to overcome gravitational forces on the fiber-forming solution, overcome surface tension forces of the solution, provide an enough force to form a stream of solution in space, and accelerate that stream across the electric field. Surface tension is a function of many variables, including the type of polymer, the solution concentration, and the temperature.

The concentration of the fiber-forming solution should be sufficiently high so that randomly coiled polymeric molecules within the solution can be oriented and form an array of molecules or a proto-filament. The concentration should be lower than the saturation limit of the polymer. However, the polymer concentration should be sufficiently low to avoid an excessively high surface tension, which would otherwise require extremely high electrical forces.

In a study of electro-spinning of silk fibers, Zarkoob, et al. (U.S. Pat. No. 6,110,590) found that a solution of from about 0.2 to about 1.3 weight percent of Nephila clavipes within hexafluroisopropanol, at room temperature and pressure, typically requires an electric field of about 24 to about 30 kV, and the distance between the liquid delivery device and the fiber collector is from about 10 to about 15 cm. Also, a solution of from about 0.6 to about 0.8 weight percent of Bombyx mori within hexafluroisopropanol, at room temperature and pressure, typically requires an electric field of from about 24 to about 30 kV, and a distance between the delivery device and the collector of about 10 to about 15 cm. These notions imply that an electric field strength of approximately 2.4 kV/m to 2,000 kV/m is required. In the presently invented electro-spinning apparatus, with an inter-electrode spacing from approximately 10 μm (or smaller) to 0.1 mm (100 μm), the required voltage would be in the range of 0.024 volts (or smaller) to 200 volts. As opposed to the conventional electro-spinning, no high voltage is required in the present apparatus. In fact, a voltage of 1-10 volts was found to be sufficient when the inter-electrode spacing is approximately 5 mm or smaller. These voltage values are well within a safe voltage range. The spinning rate can be controlled by adjusting both the flow rate of the fiber precursor solution and the electric field strength.

As shown in FIG. 1, the second electrode 26 comprises a coating on the surface of a syringe needle 36 or glass pipette. The tip of this needle or pipette has an opening or orifice 34 through which any residual solvent in the intended cavity site of a patient's body may be removed. The opposite end of this syringe needle or pipette may be connected to a vacuum pump to facilitate the removal of residual solvent.

Once the nano fibers are formed, a solution of cells, growth factors, nutrients, and/or drug may be injected, through the same or a different syringe, into the fiber scaffold in the intended site.

The nano fiber preform produced is typically in the form of a non-woven or a network of overlapping fibers with interconnected macro pores. Optionally, the same syringe (e.g., 12) or a different syringe may be used to inject a matrix polymer into the intended body site to form a composite structure. This step involves injection of a matrix polymer-forming solution into the interstitial pores of the reinforcement preform. In a simple case, this solution is composed of a polymer dissolved in a solvent. Upon injection of the solution, the solvent may be removed via pumping, leaving behind a solid polymer phase. This matrix polymer serves as a binder to bond nano fibers together at least at their points of contact, but still leaving behind macro pores which are normally interconnected. The nano fibers impart mechanical strength and stiffness to the resulting composite. Such a porous composite, comprising a nano fiber-based reinforcement phase, a polymer matrix phase, and macro pores, can be used as a scaffold for tissue regeneration.

The matrix polymer-forming solution may be mixed with desired cells (e.g., chondrocytes) and other ingredients (e.g., growth factors, nutrients, therapeutic medicine, etc.) to form a polymer-cell suspension prior to being injected. Alternatively, the cells, growth factors, nutrients, and/or drug may be injected, through the same or a different syringe, into the composite scaffold after the matrix polymer is injected.

The matrix polymer-forming solution may comprise a reactive mass (e.g., monomers, oligomers, initiators, catalysts, co-catalysts, etc.) which, upon injection into the intended site, is allowed to polymerize and/or cross-link to form a polymer gel, interpenetrating network (IPN), or semi-IPN. Directly injectable polymer precursors are known in the art (e.g., as cited earlier, U.S. Pat. No. 5,700,848 (Dec. 23, 1997); U.S. Pat. No. 5,709,854 (Jan. 20, 1998); U.S. Pat. No. 5,837,752 (Nov. 17, 1998); U.S. Pat. No. 6,129,761 (Oct. 10, 2000); U.S. Pat. No. 6,224,893 (May 1, 2001)).

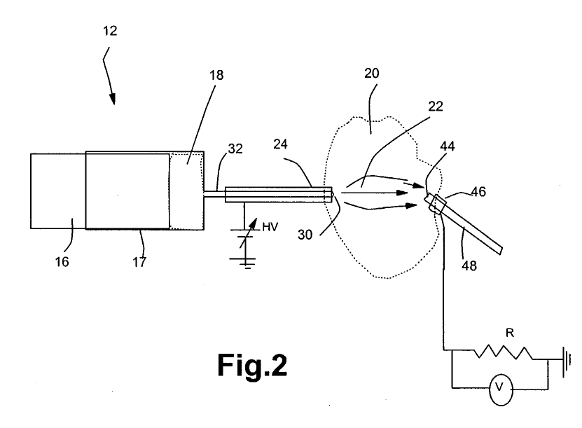

In one preferred embodiment, an optical fiber 48 may be introduced into the intended body site (FIG. 2). One end of this optic fiber is coated with a metal to serve as an electrode 46. This optic fiber may be used to transmit light through an end 44 into the intended cavity site to facilitate curing, cross-linking, and/or polymerization of the matrix polymer-forming fluid to form a polymer, co-polymer, IPN, semi-IPN, or polymer gel.

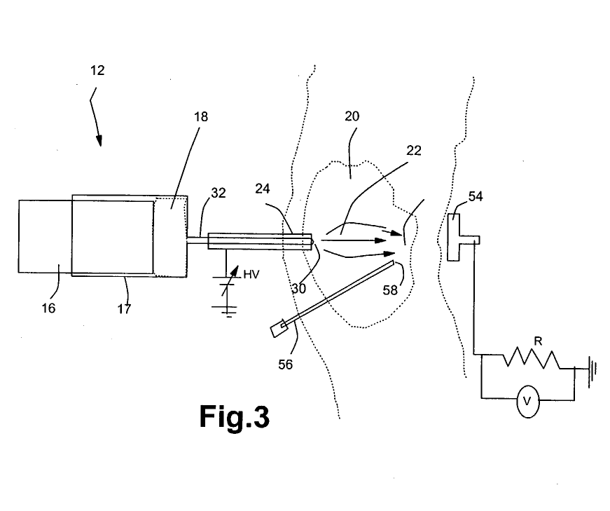

In an alternative configuration, one (e.g., 54 in FIG. 3) of the two electrodes may be placed outside of the patient's body. In this case, it may be necessary to insert a needle or pipette 56 into the intended site so that any residual solvent or undesirable volatile chemical species may be removed through the opening 58 with the assistance of a pump.

In another preferred embodiment, the injected nano fibers and matrix polymer are allowed to essentially completely fill the intended cavity site to make a solid composite. This pore-free composite is particularly useful for reinforcing a defected or cavitied bone. Many aged people are known to have highly cavitied or spongy-like bone structures, which are relatively weak. Directly injectable nano fiber composites provide a viable means of reinforcing the bone strength to reduce the risk of having a catastrophic failure or fracture.

A variety of biodegradable and absorbable (or bio-resorbable) polymers can be used to make the fiber or matrix polymer that constitutes the scaffold composite. Examples of suitable biocompatible, biodegradable, and/or bioabsorbable polymers that could be used include polymers selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, poly(anhydrides), dioxanone- and dioxepanone-based polymers, polyhydroxyalkanoates, hyaluronic acid-modified polymers, albumin-modified polymers, fibrinogen/fibrin-based materials, aliphatic carbonate-based polymers, poly(propylene fumarate), alginate hydrogels, N-isopropyleacrylamide-based polymers and gels, polyphosphazenes, biomolecules (i.e. biopolymers such as collagen, elastin, bioabsorbable starches, etc.) and blends thereof.

For use in preparing a composite scaffold, aliphatic polyesters include but are not limited to homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone, hydroxybutyrate (repeating units), hydroxyvalerate (repeating units), 1,4-dioxepan-2-one (including its dimer 1,5,8,12-tetraoxacyclotetradecane-7,14-dione), 1,5-dioxepan-2-one, 6,6-dimethyl-1,4-dioxan-2-one 2,5-diketomorpholine, pivalolactone, α,α-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof.

Polyalkylene oxalates for the purpose of this invention include those described in U.S. Pat. Nos. 4,208,511; 4,130,639; and 4,205,399. Also useful are Polyphosphazenes, co-, ter- and higher order mixed monomer based polymers made from L-lactide, D,L-lactide, lactic acid, glycolide, glycolic acid, para-dioxanone, trimethylene carbonate and ε-caprolactone. One may also select Polyanhydrides from diacids of the form HOOC—C₆H₄—O—(CH₂)ₘ—O—C₆H₄—COOH where m is an integer in the range of from 2 to 8 and copolymers thereof with aliphatic α-ω-diacids of up to 12 carbons. Polyoxaesters, polyoxaamides and polyoxaesters containing amines and/or amido groups are described in one or more of the following U.S. Pat. Nos. 5,464,929; 5,597,579; 5,618,552; 5,645,850; and 5,859,150.

Currently aliphatic polyesters are the absorbable polymers that are preferred for making composite scaffolds. Aliphatic polyesters can be homopolymers, copolymers (random, block, segmented, tappered blocks, graft, triblock, etc.) having a linear, branched or star structure. Preferred are linear copolymers. Suitable monomers for making aliphatic homopolymers and copolymers maybe selected from the group consisting of, but are not limited, to lactic acid, lactide (including L-, D-, meso and D,L mixtures), glycolic acid, glycolide, ε-caprolactone, p-dioxanone (1,4-dioxan-2-one), trimethylene carbonate (1,3-dioxan-2-one), δ-valerolactone, β-butyrolactone, ε-decalactone, 2,5-diketomorpholine, pivalolactone, α,α-diethylpropiolactone, ethylene carbonate, ethylene oxalate, 3-methyl-1,4-dioxane-2,5-dione, 3,3-diethyl-1,4-dioxan-2,5-dione, y-butyrolactone, 1,4-dioxepan-2-one, 1,5-dioxepan-2-one, 6,6-dimethyl-dioxepan-2-one, 6,8-dioxabicycloctane-7-one and combinations thereof.

The polymer or copolymer suitable for forming a composite scaffold for tissue regeneration depends on several factors. The chemical composition, spatial distribution of the constituents, the molecular weight of the polymer and the degree of crystallinity all dictate to some extent the in-vitro and in-vivo behavior of the scaffold. However, the selection of the fiber and matrix polymers to make composite scaffolds for tissue regeneration largely depends on the following factors: (a) bio-absorption (or bio-degradation) kinetics; (b) in-vivo mechanical performance; and (c) cell response to the material in terms of cell attachment, proliferation, migration and differentiation and (d) bio-compatibility.

The ability of the material substrate to resorb in a timely fashion in an intended living body environment is critical. But the differences in the absorption time under in-vivo conditions can also be the basis for combining the fiber and the matrix polymers. For example, ε-caprolactone matrix can be reinforced with poly-glycolide fiber, which is a relatively fast absorbing polymer. The L-lactide based fiber is a relatively slow absorbing polymer.

It is essential for a scaffold to be macro-porous (pore sizes greater than 100 μm) because the macro-pores provide the needed space for the cells to attach themselves to the fiber surfaces, for the cells, growth factors and nutrients to diffuse into various spots inside a scaffold, and for the metabolic waste to diffuse out. Micro-pores (pore sizes smaller than 100 μm; but preferably smaller than 10 μm) are also desirable because they provide (1) increased surface areas for cells to attach to, (2) additional parameter to control the physical density of a scaffold, (3) additional space for cells, growth factors, nutrients and metabolic waste to migrate through, and (4) enhanced bio-degradation and bio-resorption rates, if so desired.

The formation of micro pores in a matrix resin can be accomplished in several ways. For instance, a physical or chemical blowing agent may be added to the polymer forming solution. Micro pores are formed upon injection of the solution into the intended site. The preparation and activation of a physical or chemical blowing agent for polymer foaming is a well-known art. Alternatively, the micro-porous structure may be made by a polymer-solvent phase separation technique, which is carried out after injection of the matrix-polymer forming fluid (e.g., containing a polymer and a solvent). Generally, a polymer solution can be separated into two phases by any one of the four techniques: (a) thermally induced gelation/crystallization; (b) non-solvent induced separation of solvent and polymer phases; (c) chemically induced phase separation, and (d) thermally induced spinodal decomposition. The polymer solution is separated in a controlled manner into either two distinct phases or two bicontinuous phases. Subsequent removal of the solvent phase usually leaves a porous structure of density less than the bulk matrix polymer and pores in the micrometer ranges (typically 0.5 μm to 50 μm). The steps involved in the preparation of these micro pores consists of choosing the right solvents for the polymers that needs to be lyophilized and preparing a homogeneous solution. Immediately after fluid injection, the polymer solution may be subjected to a freezing and vacuum drying cycle. The freezing step phase separates the polymer solution and vacuum drying step removes the solvent by sublimation and/or drying leaving a micro-porous polymer structure.

What is claimed:

1. A process for injecting nanometer-scaled fibers directly into an intended body site of a patient, comprising:
(a) preparing a precursor fluid to said fibers, and
(b) injecting said precursor fluid into said intended body site under the influence of an electrical field of sufficient strength established between two electrodes to produce said nanometer-scaled fibers.

2. The process as defined in claim 1, wherein said nano-scaled fibers are cumulated in said intended body site to form a non-woven fiber preform shape in accordance with a shape of said intended body site.

3. The process as defined in claim 1, wherein at least one of said two electrodes comprises a device selected from the group consisting of a syringe with a metallic needle tip, a syringe with a metal-coated glass tip, a glass pipette with a metal-coated tip, or a combination thereof.

4. The process as defined in claim 1, wherein both said two electrodes comprise a device selected from the group consisting of a syringe with a metallic needle tip, a syringe with a metal-coated glass tip, a glass pipette with a metal-coated tip, or a combination thereof.

5. The process as defined in claim 1, 2, 3, or 4, wherein at least one of said electrodes is inserted inside intended body site or positioned at a desired short distance from said intended body site.

6. The process as defined in claim 5, wherein both said electrodes are inserted inside said intended body site or positioned at a short distance from said intended body site.

7. The process as defined in claim 5, wherein said two electrodes are spaced at a distance smaller than a dimension of said intended body site.

8. The process as defined in claim 5, wherein said two electrodes are spaced at a distance smaller than 10 mm.

9. The process as defined in claim 1, 2, 3, or 4, wherein at least one of said two electrodes is positioned outside said intended body site.

10. The process as defined in claim 1, 2, 3, or 4, wherein said precursor fluid comprises a fluid composition which is removed during and/or after the production of said nanometer-scaled fibers begins.

11. The process as defined in claim 10, wherein the removal of said fluid composition is facilitated by using a pump means.

12. The process as defined in claim 1, 2, 3, or 4, further comprising a step of injecting cells, growth factors, nutrients, and/or therapeutic drug into said intended body site.

13. The process as defined in claim 2, wherein said fiber preform comprises macro pores which are essentially interconnected.

14. The process as defined in claim 1, wherein the nanometer-scaled fibers are biodegradable, bio-compatible, and/or bio-resorbable.

15. The process as defined in claim 1, 2, 3, 4, or 13, further comprising an additional step of injecting a matrix polymer-forming fluid into said intended body site to form a matrix polymer therein, wherein said matrix polymer and said nanometer-scaled fibers combine to form a composite structure.

16. The process as defined in claim 15, wherein said composite structure comprises a network of interconnected macro pores in which cells grow and proliferate.

17. The process as defined in claim 15, wherein the nanometer-scaled fibers, the matrix polymer, or both are biodegradable, bio-compatible, and/or bio-resorbable.

18. The process as defined in claim 15, wherein said matrix polymer-forming fluid comprises a suspension of dissociated cells in a solution of a biocompatible polymer precursor and said dissociated cells are injected into said intended body site along with said polymer precursor.

19. The process as defined in claim 15, wherein said matrix polymer-forming fluid comprises a volatile liquid which is injected into said intended body site along with said matrix polymer and wherein said process further includes a step of removing said volatile liquid upon injection into said body site.

20. The process as defined in claim 15, wherein said matrix-forming fluid further comprises growth factors and/or nutrients to promote cell growth, proliferation and/or differentiation.

21. The process as defined in claim 15, wherein said matrix polymer is selected from the group consisting of aliphatic polyesters, poly(amino acids), copoly(ether-esters), polyalkylenes oxalates, polyamides, poly(iminocarbonates), polyorthoesters, polyoxaesters, polyaminoesters, polyoxaesters containing amine groups, poly(anhydrides), dioxanone- and dioxepanone-based polymers, polyhydroxyalkanoates, hyaluronic acid-modified polymers, albumin-modified polymers, fibrinogen/fibrin-based materials, aliphatic carbonate-based polymers, poly(propylene fumarate), alginate hydrogels, N-isopropylacrylamide-based polymers and gels, polyphosphazenes, poly(caprolactone), collagen, elastin, starches, chitosan, and blends or co-polymers thereof.

22. The process as defined in claim 1 or 15, wherein said nanometer-scaled fibers comprise a material composition selected from the group consisting of aliphatic polyesters, poly(amino acids), copoly(ether-esters), polyalkylenes oxalates, polyamides, poly(iminocarbonates), polyorthoesters, polyoxaesters, polyaminoesters, polyoxaesters containing amine groups, poly(anhydrides), dioxanone- and dioxepanone-based polymers, polyhydroxyalkanoates, hyaluronic acid-modified polymers, albumin-modified polymers, fibrinogen/fibrin-based materials, aliphatic carbonate-based polymers, poly(propylene fumarate), alginate-containing polymers, N-isopropylacrylamide-based polymers and gels, polyphosphazenes, poly(caprolactone), collagen, elastin, starches, chitosan, and blends or co-polymers thereof.

23. The process as defined in claim 15, wherein said matrix polymer comprises a material composition selected from the group consisting of a polymer gel, a semi-interpenetrating network, an interpenetrating network, or a combination thereof.

24. The process as defined in claim 15, wherein said matrix polymer-forming fluid comprises a foaming agent for forming micro-pores in said composite structure.

25. The process as defined in claim 15, wherein said matrix polymer-forming fluid comprises a suspension of dissociated cells in a solution of a biocompatible polymer which is injected into said intended body site, wherein the polymer crosslinks upon exposure to free radicals to form a hydrogel, and wherein the process further includes a step exposing the suspension in situ in said intended site to free radicals generated by electromagnetic radiation from an electromagnetic source external to the injected suspension, the electromagnetic radiation penetrates through tissue to generate the free radicals which cause polymer crosslinking and whereby the composite structure forms.

26. The process as defined in claim 15, wherein said matrix polymer-forming fluid comprises a suspension of dissociated cells in a solution of a biocompatible polymer which is injected into said intended body site, wherein the polymer crosslinks upon exposure to free radicals to form a hydrogel, and wherein the process further includes a step exposing the suspension in situ in said intended site to free radicals generated by electromagnetic radiation from an electromagnetic source external to the injected suspension, the electromagnetic radiation is introduced through an optical fiber, glass pipette, or a waveguide to generate the free radicals which cause polymer cross-linking and whereby the composite structure forms.
